(19) United States
(12) Patent Application Publication
(10) Pub. No.: US 2002/0128264 A1
(43) Pub. Date: Sep. 12, 2002

(54) Methods for treatment of conditions affected by activity of multidrug transporters

(76) Inventor: Eve M. Taylor, Del Mar, CA (US)

Correspondence Address: OPPENHEIMER WOLFF & DONNELLY LLP, Attn: Michael B. Farber, Esq., Suite 3800, 2029 Century Park East, Los Angeles, CA, 90067, US

(21) Appl. No.: 09/900,297

(22) Filed: Jul. 6, 2001

Related U.S. Application Data
(60) Provisional application No. 60/216,616, filed on Jul. 7, 2000.

(51) Int. Cl. A61K 31/541; A61K 31/5377; A61K 31/522; A61K 31/496

(52) U.S. Cl. 514/228.2; 514/263.35; 514/234.5; 514/252.16; 514/263.3; 514/256; 514/419

(57) ABSTRACT

One aspect of the present invention is a method of treating a condition or disease associated with the activity of a multidrug transporter protein comprising administering to a mammal with a condition or disease associated with the activity of a multidrug transporter protein an effective quantity of a purine derivative or analogue, a tetrahydroindolone derivative or analogue, or a pyrimidine derivative or analogue. If the compound is a purine derivative, the purine moiety can be guanine or hypoxanthine. A particularly preferred bifunctional purine derivative is N-4-carboxyphenyl-3-(6-oxohydropurin-9-yl) propanamide. Methods according to the present invention can be used to treat cancer, a microbial or parasitic infection, HIV, infection, or a condition associated with inflammation such as asthma or rheumatic disease.

### CROSS-REFERENCES

This application claims priority from Provisional Application Serial No. 60/216,616, filed Jul. 7, 2000, by Eve M. Taylor, and entitled “Methods for Treatment of Conditions Affected by Activity of Multidrug Transporters,” which is incorporated herein in its entirety by this reference.

### BACKGROUND OF THE INVENTION

This invention is directed to methods for treatment of conditions affected by activity of multidrug transporters, particularly with purine derivatives or analogues, tetrahydroindolone derivatives or analogues, or pyrimidine derivatives or analogues.

Multidrug transporters are membrane proteins that are able to expel a broad range of toxic molecules from the cell. These multidrug transporters belong to the ATP-binding cassette (ABC) family of transport proteins that utilize the energy of ATP hydrolysis for activity.

Many cancers are intrinsically resistant to anticancer drugs or become resistant to chemotherapy after many rounds of treatment. A major mechanism of resistance of cancer cells to cancer drugs such as adriamycin, etoposide, vinblastine, actinomycin D, and taxol is the overexpression of the ABC transporters, P-glycoprotein and the Multidrug Resistance Associated Proteins (MRPs).

In microorganisms, multidrug transporters play an important role in conferring antibiotic resistance on pathogens. Homologues of human P-glycoprotein and MRP have been found in microorganisms and have been implicated in malaria drug resistance. Multidrug transporters found in bacteria are described in H. W. van Veen & W. I. Konings, “Multidrug Transporters from Bacteria to Man: Similarities in Structure and Function,” Semin. Cancer Res. 8: 188-191 (1997).

MDR transporters are expressed in normal tissues including kidney, intestine, brain, liver, testes, and placenta. They function in the efflux of compounds from these organs and thus influence the absorption, the excretion, and the body distribution of drugs. For example, multidrug transporters have been shown to confer low brain penetration of antiviral compounds used to treat HIV reducing their efficacy in the treatment of AIDS-related neurodegeneration.

In addition to a role in the efflux of drugs, MRPs transport endogenous leukotriene C₄ (LTC₄). LTC₄ is an active component of the “slow reacting substance of anaphylaxis” and is implicated in the pathogenesis of inflammatory diseases such as asthma and rheumatic disease.

In addition to P-glycoprotein and MRPs, this class of transporters includes the lung-resistance protein (LRP) and the transporter of antigenic peptides (TAP). These transporters are described in the following references: M. M. Gottesman & I. Pastan, “Biochemistry of Multidrug Resistance Mediated by the Multidrug Transporter”, Annu. Rev. Biochem. 63: 385-427 (1993); V. Ling, “Multidrug Resistance: Molecular Mechanisms and Clinical Relevance”, Cancer Chemother. Pharmacol. 40 (Suppl): S3-S8 (1997); F. J. Sharom, “The P-Glycoprotein Efflux Pump: How Does It Transport Drugs?”, J. Membrane Biol. 160: 161-175 (1997); A. F. List, “Non-P-Glycoprotein Drug Export Mechanisms of Multidrug Resistance”, Semin. Hematol. 34 (Suppl. 5): 20-24 (1997); W. A. Banks, “Physiology and Pathology of the Blood-Brain Barrier: Implications for Microbial Pathogenesis, Drug Delivery and Neurodegenerative Disorders”, J. Neurovirol. 5: 538-555 (1999); A. Lo & G. J. Burckart, “P-Glycoprotein and Drug Therapy in Organ Transplantation”, J. Clin. Pharmacol. 39: 999-1005 (1999); and D. Lautieret al., “Multidrug Resistance Mediated by the Multidrug Resistance Protein (MRP) Gene”, Biochem. Pharmacol. 52: 967-977 (1996); P. Borst, R. Evers, M. Kool & J. Wijnholds, “The multidrug resistance protein family”, Biochim. Biophys. Acta 1461: 347-357.

Therefore, there exists a need for methods that can modulate or inhibit the activity of multidrug transporter proteins to enable improved treatment of cancer, infectious diseases, HIV infection, and inflammation, among other diseases and conditions in which the activity of multidrug transporter proteins is significant. Preferably, these methods should be able to be combined with methods that enable active compounds to pass through the blood-brain barrier, making combined therapy more efficient. These methods should also be suitable for use with a large variety of active compounds and should not depend on the specific interactions between each active compound and the transporter proteins.

### SUMMARY

One embodiment of the present invention is a method of treating a condition or disease associated with the activity of a multi-drug transporter protein comprising administering to a patient suffering from a condition or disease associated with the activity of a multi-drug transporter protein an effective quantity of a compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L though a carbonyl group wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

The purine moiety can be selected from the group consisting of hypoxanthine and guanine, as well as other purine moieties. A number of purine derivatives suitable for use in methods according to the present invention are disclosed. A particularly preferred purine derivative is N-4-carboxyphenyl-3-(6-oxohydropurin-9-yl)propanamide. Preferably, the compound is capable of passing through the blood-brain barrier.

The multidrug transporter protein can be selected from the group consisting of P-glycoprotein and multidrug resistance associated proteins (MRPs).

The condition or disease associated with the activity of a multidrug transporter protein can be selected from the group consisting of cancer, a microbial or parasitic infection, HIV infection, and a condition associated with inflammation. The condition associated with inflammation can be asthma or rheumatic disease. (but are not limited to)

Another embodiment of the present invention is a method of increasing intestinal absorption of a drug transported by a multidrug transporter protein comprising administering to a mammal an effective quantity of a bifunctional purine derivative capable of bypassing the blood-brain barrier as described above.

Yet another embodiment of the present invention is a method of improving the penetration of a drug transported by a multidrug transporter into the central nervous system comprising administering to a mammal an effective quantity of a compound as described above.

Still another embodiment of the present invention is a method of decreasing renal excretion or renal toxicity of a drug transported by a multidrug transporter protein comprising administering to a mammal an effective quantity of a compound as described above.

Yet another embodiment of the present invention is a method of treating a malignancy comprising:

(1) administering an effective quantity of an antineoplastic agent transported by a multidrug transporter protein to a mammal with cancer; and

(2) administering an effective quantity of a compound as described above.

In this method, the antineoplastic agent can be selected from the group consisting of adriamycin, etoposide, vinblastine, actinomycin D, and taxol.

Yet another embodiment of the present invention is a method for screening a compound for the existence or nonexistence of multidrug resistance transporter protein inhibitory activity comprising the steps of:

(1) adding the compound to a culture of cancer cells that constitutively express or are induced to express at least one multidrug resistance transporter protein;

(2) adding a cytotoxic agent transported by the multidrug resistance transport protein to the cells;

(3) determining the effect of the compound on the activity of the multidrug resistance transporter protein by performing one or both of a cytotoxicity assay and a drug accumulation assay on the cancer cells to measure either the cytotoxicity of the cytotoxic agent or the accumulation of the cytotoxic agent in the cancer cells; and

(4) comparing the effect of the compound on the activity of the multidrug transporter protein with the effect of a reference compound, N-4-carboxyphenyl-3-(6-oxohydropurin-9-yl)propanamide.

### BRIEF DESCRIPTION OF THE DRAWINGS

The following invention will become better understood with reference to the specification, appended claims, and accompanying drawings, where:

FIG. 1 is a set of graphs showing the efflux of ¹⁴C-labeled N-4-carboxyphenyl-3-(6-oxohydropurin-9-yl)propanamide (AIT-082) and ³H-labeled sucrose after intracerebroventricular (icv) administration: (a) graph showing time course; (b) bar graph showing t_{½};

FIG. 2 is a set of graphs showing the efflux of ¹⁴C-labeled AIT-082 and ³H-labeled sucrose after intraparenchymal (ipc) administration: (a) graph showing time course; (b) bar graph showing t₁₂;

FIG. 3 is a graph showing the saturability of ¹⁴C-labeled AIT-082 efflux;

FIG. 4 is a graph showing the effect of inhibitors of P-gp and MRP on the efflux of ¹⁴C-labeled AIT-082 after icv administration;

FIG. 5 is a graph showing the effect of inhibitors of P-gp and MRP on the efflux of ¹⁴C-labeled AIT-082 after ipc administration; and

FIG. 6 is a graph showing the effect of AIT-082 on the efflux of ³H-labeled quinidine after icv administration.

### DESCRIPTION

We have discovered that the bifunctional purine derivative N-4-carboxyphenyl-3-(6-oxohydropurin-9-yl)propanamide (also known as AIT-082 and leteprinim potassium), which bypasses the blood-brain barrier and is transported into brain by a nonsaturable mechanism, is an inhibitor or modulator of the multidrug transporters P-glycoprotein and MRP. This property of inhibiting or modulating one or more of the multidrug transporters P-glycoprotein and MRP, therefore, should also be possessed by other purine derivatives and analogues, tetrahydroindolone derivatives and analogues, and pyrimidine derivatives and analogues that can pass through the blood-brain barrier, as discussed below.

Accordingly, one aspect of the present invention is a method of treating a condition or disease associated with the activity of a multidrug transporter protein comprising administering to a mammal with a condition or disease associated with the activity of a multidrug transporter protein an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L though a carbonyl group wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

As used herein, the term “associated with” means either than the disease or condition directly affects the activity of a multidrug transporter protein, such as occurs in the overproduction of these proteins in cancer cells, or that, in the absence of inhibition of the multidrug transporter protein, the activity of the protein affects the treatment of the disease or condition such as by causing accelerated transport of a drug out of cells that are affected by the disease or condition.

Typically, a compound useful in a method of the present invention is capable of passing through the blood-brain barrier.

In one preferred embodiment of methods according to the present invention, the moiety A is a purine moiety.

In one alternative, A is a substituted or unsubstituted hypoxanthine moiety. Typically, in this alternative, L has the structure —(CH₂)ₙ— where n is an integer from 1 to 6.

The compound can be a compound of formula (I)

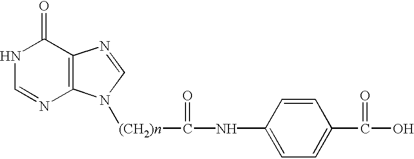

where n is an integer from 1 to 6 and R is hydrogen or lower alkyl or is a salt or prodrug ester of a compound of formula (I) wherein n is an integer from 1 to 6 and R is hydrogen or lower alkyl. Typically, the compound is a compound of formula (I) wherein n is an integer from 1 to 6 and R is hydrogen or lower alkyl. Typically, R is hydrogen, and the compound is N-4-[[3-(6-oxo-1,6-dihydropurin-9-yl)-1-oxopropyl]amino]benzoic acid, designated AIT-082. Alternatively, R is ethyl, and the compound is N-4-[[3-(6-oxo-1,6-dihydropurin-9-yl)-1-oxopropyl]amino]benzoic acid ethyl ester.

When the purine moiety is hypoxanthine, a preferred purine derivative is a compound of formula (I)

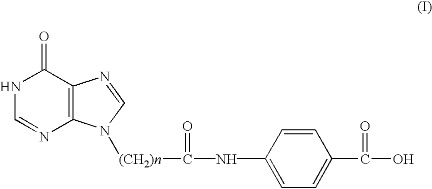

wherein n is an integer from 1 to 6 or of a salt or prodrug ester of formula (I) wherein n is an integer from 1 to 6. Typically, the purine derivative is a compound of formula (I) wherein n is an integer from 1 to 6. Preferably, n is 2 and the compound is N-4-carboxyphenyl-3-(6-oxohydropurin-9-yl)propanamide, also known as AIT-082. The activity of this compound is described further in the Example.

Alternatively, the purine derivative can be a 9-substituted hypoxanthine derivative of formula (II)

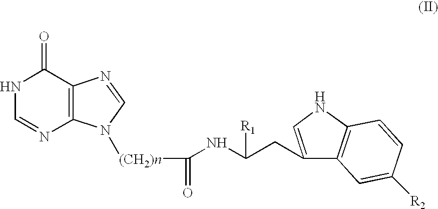

wherein n is a integer from 1 to 6, R₁ is selected from the group consisting of H, COOH, and COOW₁, where W₁ is selected from the group consisting of lower alkyl, amino, and lower alkylamino, and R₂ is selected from the group consisting of H and OH.

In this alternative, for one particularly preferred purine derivative, n is 2, R₁ is H and R₂ is OH and the purine derivative is N-(2-(5-hydroxyindol-3-yl))ethyl-3-(6-oxohydropurine-9-yl)propanamide. In this alternative, for another particularly preferred purine derivative, n is 2, R₁ is H and R₂ is H and the purine derivative is N-(2-indol-3-yl)ethyl-3-(6-oxohydropurin-9-yl)propanamide. In this alternative, for still another particularly preferred purine derivative, n is 2, R₁ is COOH, and R₂ is OH and the purine derivative is N-(1-carboxyl-(2-(5-hydroxyindol-3-yl))ethyl-3-(6-oxohydropurin-9-yl) propanamide.

As another alternative, the purine derivative can be a 9-substituted hypoxanthine derivative of formula (III)

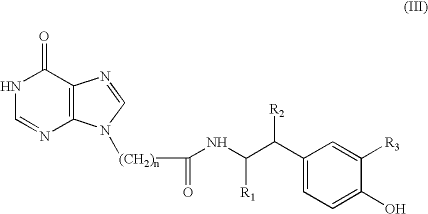

wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

In this alternative, for one particularly preferred purine derivative, n is 2, R₁ is H, R₂ is H, and R₃ is OH, and the purine derivative is N—(2-(3,4-dihydroxyphenyl))ethyl-3-(6-oxohydropurin-9-yl)propanamide. In this alternative, for another particularly preferred purine derivative, n is 2, R₁ is H, R₂ is OH, and R₃ is OH, and the purine derivative is N—(2-hydroxy-2-(3,4-dihydroxyphenyl))ethyl-3-(6-oxohydropurin-9-yl) propanamide. In this alternative, for still another particularly preferred purine derivative, n is 2, R₁ is COOH, R₂ is H, and R₃ is OH, and the purine derivative is N—(1-carboxyl-2-(3,4-dihydroxyphenyl))ethyl-3-(6-oxohydropurin-9-yl)propanamide.

When the purine moiety is guanine, one preferred purine derivative is a 9-substituted guanine derivative of formula (IV)

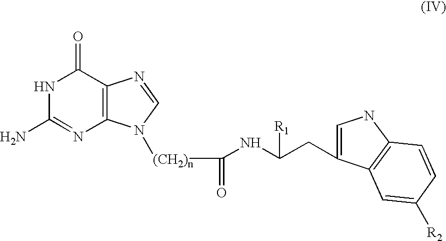

wherein n is an integer from 1 to 6, R₁ is selected from the group consisting of H, COOH, and COOW₁, or W₁ is lower alkyl, amino, or lower alkylamino, and R₂ is selected from the group consisting of H and OH.

In this alternative, for one particularly preferred purine derivative, n is 2, R₁ is H, and R₂ is OH, and the purine derivative is N—(2-(5-hydroxindol-3-yl))ethyl-3-(2-amino-6-oxohydropurin-9-yl)propanamide. In this alternative, for another particularly preferred purine derivative, n is 2, R₁ is H, and R₂ is H and the purine derivative is N—(2-(2-indol-3-yl)ethyl))-3-(2-amino-6-oxohydropurin-9-yl))propanamide. In this alternative, for still another particularly preferred purine derivative, n is 2, R₁ is COOH, and R₂ is OH, and the purine derivative is N—(1-carboxyl)-(2-(5-hydroxyindol-3-yl))ethyl-3-(2-amino-6-oxohydropurin-9-yl)propanamide.

Alternatively, the purine derivative can be a 9-substituted guanine derivative of formula (V) wherein n is an integer from 1 to 6.

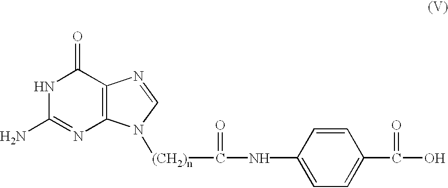

In this alternative, for one particularly preferred purine derivative, n is 2 and the compound is N-4-carboxyphenyl-3-(2-amino-6-oxohydropurin-9-yl)propanamide.

Alternatively, the purine derivative can be a 9-substituted guanine derivative of formula (VI) wherein n is an integer from 1 to 6.

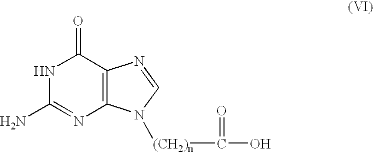

In this alternative, for one particularly preferred purine derivative, n is 2 and the compound is 3-(2-amino-6-oxohydropurine-9-yl)propanoric acid.

Alternatively, the purine derivative can be a 9-substituted guanine derivative of formula (VII) wherein n is an in integer from 1 to 6, p is an integer from 1 to 6, and q is an integer from 1 to 3.

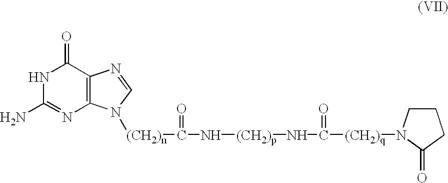

In this alternative, for one particularly preferred purine derivative, n is 2, p is 2, and q is 1, and the purine derivative is N-[2-[[2-(2-oxopyrrolidin-1-yl)-1-oxoethyl]amino]ethyl]propanamide.

Alternatively, the purine derivative can be a 9-substituted guanine derivative of formula (VIII) wherein R₁ is selected from the group consisting of H, COOH, and COOW₁, where W₁ is selected from the group consisting of lower alkyl, amino, and lower alkylamino, R₂ is selected from the group consisting of H and OH, and R₃ is selected from the group consisting of H and OH.

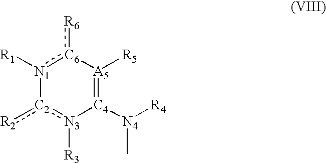

In this alternative, for one particularly preferred purine derivative, n is 2, R₁ is H, R₂ is H, and R₃ is OH, and the purine derivative is N—(2-(3,4-dihydroxyphenyl)ethyl-3-(2-amino-6-oxohydropurin-9-yl)propanamide. In this alternative, for another particularly preferred purine derivative, n is 2, R₁ is H, R₂ is OH, and R₃ is OH, and the purine derivative is N-(2-hydroxy-2-(3,4-dihydroxyphenyl)ethyl)-3-(2-amino-6-oxohydropurin-9-yl)propanamide. In this alternative, for still another particularly preferred purine derivative, n is 2, R₁ is COOH, R₂ is H, and R₃ is H and the compound is N-(1-carboxyl-2-(3,4-dihydroxyphenyl)ethyl)-3-(2-amino-6-oxohydropurin-9-yl)propanamide.

Alternatively, the purine derivative can be a 9-substituted guanine derivative of formula (IX) wherein n is an integer from 1 to 6 and p is an integer from 1 to 3.

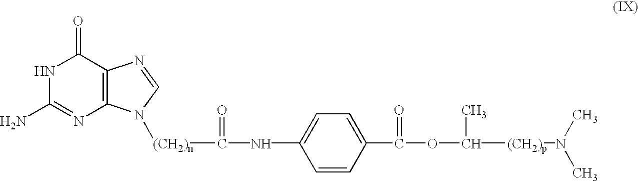

In this alternative, for one particularly preferred purine derivative, n is 2, p is 1, and the compound is the 1-(dimethylamino)-2-propyl ester of N-4-carboxyphenyl-3-(2-amino-6-oxohydropurin-9-yl)propanamide.

Other bifunctional hypoxanthine derivatives suitable for use in methods according to the present invention are disclosed in U.S. Pat. No. 5,091,432 to Glasky, incorporated herein by this reference. Other bifunctional guanine derivatives suitable for use in methods according to the present invention are disclosed in U.S. patent application Ser. No. 09/419,153, by Glasky et al., incorporated herein by this reference.

More generally, purine-based compounds suitable for use in methods according to the present invention are compounds in which A is a substituted or unsubstituted 9-atom bicyclic moiety in which the 5-membered ring has 1 to 3 nitrogen atoms, the bicyclic moiety having the structure of formula (X)

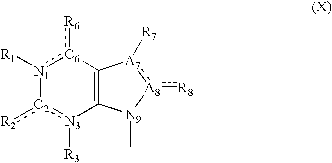

where:

(1) if the bond between N₁ and the bond between C₅ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) A₇ and A₈ are C or N;

(a) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;

(b) if A₇ and A₈ are both C and the bond between A₇ and A8 is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;

(c) if A₇ and A₈ are both N, then the bond between A₇ and A₈ is a double bond, and R₇ and R₈ are not present;

(d) if A₇ is C and A₈ is N, then the bond between A₇ and A₈ is a double bond, R₇ is hydrogen, and R₈ is not present;

(e) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;

(f) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and

(6) N₉ is bonded to L; with the proviso that A does not have the structure of an unsubstituted guanine or hypoxanthine.

The purine moiety can be a purine moiety of formula (XI)

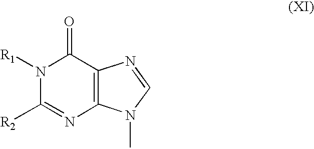

in which:

(1) R₁ is selected from the group consisting of hydrogen, alkyl, aralkyl, cycloalkyl, and heteroaralkyl; and

(2) R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylokoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

The purine moiety of formula (XI) is a hypoxanthine or a guanine derivative but excludes unsubstituted hypoxanthine, in which R₁ and R₂ are hydrogen, and unsubstituted guanine, in which R₁ is hydrogen and R₂ is amino.

In one particularly preferred embodiment, R₁ is butyl and R₂ is hydrogen.

In another preferred embodiment, R₁ is benzyl and R₂ is hydrogen.

In another preferred embodiment, R₁ is dimethylaminoethyl and R₂ is hydrogen.

In another preferred embodiment, R₁ is cyclopentyl and R₂ is hydrogen.

In another preferred embodiment, R₁ is cyclohexylmethyl and R₂ is hydrogen.

In another preferred embodiment, R₁ is cyclopropylmethyl and R₂ is hydrogen.

In another preferred embodiment, R₁ is hydrogen and R₂ is phenyl.

In another preferred embodiment, R₁ is hydrogen and R₂ is trifluoromethyl.

In another preferred embodiment, R₁ is hydrogen and R₂ is butyl.

In another preferred embodiment, R₁ is butyl and R₂ is butyl.

In another preferred embodiment, R₁ is hydrogen and R₂ is methyl.

In another preferred embodiment, R₁ is hydrogen and R₂ is phenylamino.

Alternatively, the purine moiety can be a purine moiety of Formula (XII)

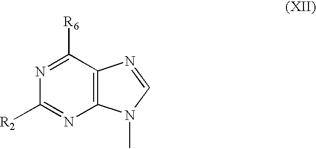

in which:

(1) R₂ is selected from the group consisting of hydrogen, halo, amino, OQ₃, SQ₃, NHNH₂, NHOQ₃, NQ₃O₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(2) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

In one preferred example of this embodiment, R₂ is hydrogen and R₆ is —NH₂ or —N(CH₃)₂.

In another preferred example of this embodiment, R₂ is hydrogen and R₆ is Cl.

In yet another preferred example of this embodiment, R₂ is —NH₂ and R₆ is Cl.

In another alternative, the purine moiety is the purine moiety of Formula (XIII)

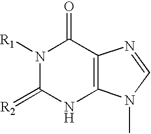

in which:

(1) R₁ is hydrogen, alkyl, aralkyl, cycloalkyl, or heteroaralkyl; and

(2) R₂ is O or S.

Preferably, in this embodiment, R₁ is hydrogen and R₂ is O or S.

Particularly preferred purine-based compounds for use in methods according to the present invention include: (1) 4-[3-(1-benzyl-6-oxo-1,6-dihydropurin-9-yl)propionylamino]benzoic acid ethyl ester; (2) 4-[3-(1-butyl-6-oxo-1,6-dihydropurin-9-yl)propionylamino]benzoic acid ethyl ester; (3) 4-[3-(1 -methyl-6-oxo-1,6-dihydropurin-9-yl)propionylamino]benzoic acid ethyl ester; (4) 4-[3-(1-(2-dimethylaminoethyl)-6-oxo-1,6-dihydropurin-9-yl)propionylamino]benzoic acid ethyl ester; (5) 4-[3-(2,6-dioxo-1,2,3,6-etrahydropurin-9-yl )propionylamino]benzoic acid ethyl ester; (6) 4-[3-(6-methoxypurin-9-yl)propionylamino]benzoic acid ethyl ester; (7) 4-[3-(6-dimethylaminopurin-9-yl)propionylamino]benzoic acid ethyl ester; (8) 4-[3-(2-amino-6-chloropurin-9-yl)propionylamino]benzoic acid ethyl ester; (9) 4-[2-(6-oxo-2-thioxo-1,2,3,6-tetrahydropurin-9-yl)propionylamino]benzoic acid ethyl ester; (10) 4-[2-(2-butyl-6-oxo-1,6-dihydropurin-9-yl)propionylamino]benzoic acid ethyl ester; (11) 4-[2-(6-oxo-2-phenyl-1,6-dihydropurin-9-yl)propionylamino]benzoic acid ethyl ester; (12) 4-{[3-(6-chloropurin-9-yl)propionyl]methylamino}benzoic acid methyl ester; (13) 3-(1 -benzyl-6-oxo-1,6-dihydropurin-9-yl)-N-[3-(2-oxopyrrolidin-1 -yl)propyl]propionamide; (14) 3-(1-benzyl-6-oxo-1,6-dihydropurin-9-yl)-N-{2-[2-(2-oxopyrrolidin-1 -yl)acetylamino]ethyl}propionamide; (15) N-3-(2-oxopyrrolidin-1-yl)propyl]-3-(6-oxo-2-thioxo-1,2,3,6-tetrahydropurin-9-yl)propionamide; and (16) 3-(1-benzyl-6-oxo-1,6-dihydropurin-9-yl)-N-(3-morpholin-4-yl-propyl)propionamide.

In another alternative of methods according to the present invention, the compound is a tetrahydroindolone derivative or analogue where A is a 9-atom bicyclic moiety in which the 5-membered ring has one to three nitrogen atoms, the bicyclic moiety having the structure of formula (XIV)

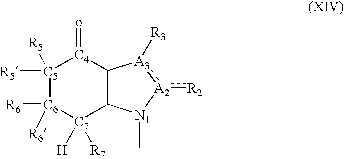

where:

(1) N₁ is bonded to L;

(2) A₂ and A₃ are C or N;

(a) If A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;

(b) If A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;

(c) If A₂ and A₃ are both N, then the bond between A₂ and A₃ is a double bond and R₂ and R₃ are not present;

(d) If A₂ is N and A₃ is C, then the bond between A₂ and A₃ is a double bond, R₂ is not present, and R₃ is hydrogen;

(e) If A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;

(f) If A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;

(3) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(4) R₅ is hydrogen unless R₅ is alkyl, in which case R₅ is hydrogen or the same alkyl as R₅;

(5) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;

(6) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(7) R_{6′} is hydrogen unless R₆ is alkyl, in which case R_{6′} is hydrogen or the same alkyl as R₆;

(8) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S; and

(9) R₇ is hydrogen unless R₅ is alkyl and R_{5′} is hydrogen, in which case R₇ is the same alkyl as R₅.

Typically, A is a tetrahydroindolone moiety. More typically, the tetrahydroindolone moiety is a tetrahydroindolone moiety of formula (XV)

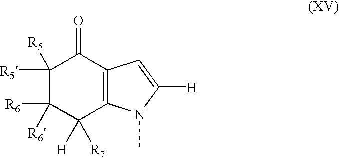

in which:

(1) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;

(2) R₅ is hydrogen;

(3) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;

(4) R₆ is hydrogen; and

(5) R₇ is hydrogen.

Typically, R₅, R_{5′}, R₆, R_{6′}, and R₇ are all hydrogen.

When A is a tetrahydroindolone moiety, preferred compounds are 4-[3-(4-oxo-4,5,6,7-tetrahydroindolon-1-yl)propionylamino]benzoic acid ethyl ester and 4-[3-(4-oxo-4,5,6,7-tetrahydroindolon-1-yl)propionylamino]benzoic acid.

In another alternative, the compound is a pyrimidine derivative or pyrimidine analogue. In this alternative, A is is an amino-substituted 6-membered heterocyclic moiety of formula (XVI)

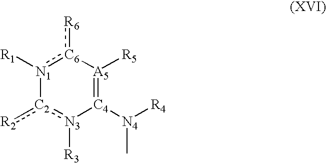

where:

(1) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;

(6) A₅ is carbon or nitrogen;

(7) if A₅ is nitrogen, then R₅ is not present;

(8) if A₅ is carbon, then R₅ is hydrogen, amino, alkyl, alkoxy, halo, nitro, aryl, cyano, alkenyl, or alkaryl;

(9) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(10) N₄ is bonded to L.

Typically, A₅ is carbon and the 6-membered heterocyclic moiety is a pyrimidine moiety.

When A is a pyrimidine moiety, in one alternative, R₂ is O and R₃ is hydrogen. In this alternative, the pyrimidine moiety can be cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, or 5-hydroxymethyluracil.

In another alternative, R₂ is S and R₃ is hydrogen. In this alternative, the pyrimidine moiety can be 2-thiouracil, 5-methylamino-2-thiouracil, 5-methyl-2-thiouracil, or 2-thiocytosine.

In still another alternative, R₂ is amino and the bond between C₂ and N₃ is a double bond. In this alternative, the pyrimidine moiety can be 2-aminopyrimidinone or 2-amino-4-chloropyrimidine.

In still another alternative, R₂ is hydrogen and the bond between C₂ and N₃ is a double bond. In this alternative, the pyrimidine moiety can be 4-chloropyrimidine, 5-amino-4-chloropyrimidine, 4-chloro-5-methylpyrimidine, 4-chloro-5-hydroxymethylpyrimidine, or 4-chloro-5-carboxymethylpyrimidine.

In still another alternative, R₁ is hydrogen, methyl, or ethyl, R₅ is hydrogen, methyl, or ethyl, and R₆ is O. In this alternative, the pyrimidine moiety can be pyrimidinone.

Particularly preferred pyrimidine compounds include: 4-[3-(2-amino-6-chloropyrimidin-4-ylamino)propionylamino]benzoic acid ethyl ester; 4-[3-(5-amino-6-chloropyrimidin-4-ylamino)propionylamino]benzoic acid ethyl ester; 4-[3-(6-chloropyrimidin-4-ylamino)propionylamino]benzoic acid ethyl ester; 4-[3-(2-amino-6-chloropyrimidin-4-ylamino)propionylamino]benzoic acid; 4-[3-(6-chloropyrimidin-4-ylamino)propionylamino]benzoic acid; 4-[3-(5-amino-6-chloropyrimidin-4-ylamino)propionylamino]benzoic acid; 3-[3-(2 -amino-6-chloropyrimidin-4-ylamino)propionylamino]benzoic acid ethyl ester; 3-[3-(6-chloropyrimidin-4-ylamino)propionylamino]benzoic acid ethyl ester; 3-[3-(5-amino-6-chloropyrimidin-4-ylamino)propionylamino]benzoic acid ethyl ester; 3-[3-(2-amino-6-chloropyrimidin-4-ylamino)propionylamino]benzoic acid; 3-[3-(6-chloropyrimidin-4-ylamino)propionylamino]benzoic acid; and 3-[3-(5-amino-6-chloropyrimidin-4-ylamino)propionylamino]benzoic acid.

In accordance with the present invention, and as used herein, the following terms, when appearing alone or as part of a moiety including other atoms or groups, are defined with the following meanings, unless explicitly stated otherwise. In addition, all groups described herein can be optionally substituted unless such substitution is excluded. The term “alkyl,” as used herein at all occurrences, refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “alkenyl,” as used herein at all occurrences, refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “alkoxy” refers to the ether —O-alkyl, where alkyl is defined as as above. The term “aryl” refers to aromatic groups which have at least one ring having a conjugated π-electron system and includes carbocyclic aryl and biaryl, both of which may be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above. The term “sulfonyl” refers to the group —S(O₂)—. The term “alkanoyl” refers to the group —C(O)Rg, where Rg is alkyl. The term “aroyl” refers to the group —C(O)Rg, where Rg is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group. As used herein, the term “lower,” in reference to an alkyl or the alkyl portion of an another group including alkyl, is defined as a group containing one to six carbon atoms. The term “optionally substituted” refers to one or more substituents that can be lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo. The term “halo” refers generally to fluoro, chloro, bromo, or iodo; more typically, “halo” refers to chloro.

As indicated above, the linker L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo. Preferably, the linker L has the structure —(CH₂)ₙ— wherein n is an integer from 1 to 6. As detailed below, for most preferred embodiments of compounds useful in methods according to the present invention, a preferred linker has n equal to 2 or 3.

The moiety B is either: (i) —OZ, where Z is hydrogen, alkyl, aryl, heteroaryl, cycloalkyl, aralkyl, or heteroaralkyl; or (ii) N(Y₁)-D, where D is a moiety that promotes absorption of the compound, and Y₁ is hydrogen, alkyl, aryl, heteroaryl, aralkyl, heteroaralkyl, which, when taken with D, can form a cyclic 5- or 6-membered saturated ring which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen. Where the moiety B is —OZ, the moiety B is a carboxylic acid or carboxylic acid or ester. Typically, where B is a carboxylic acid ester, the moiety Z is a lower alkyl, such as methyl, ethyl, butyl, propyl, or isopropyl.

In one alternative, the moiety D, as described above, is a moiety having at least one polar, charged, or hydrogen-bond-forming group to improve the metabolic and bioavailability properties of the compound. The moiety D can be, but is not limited to, a moiety with physiological or biological activity such as nootropic activity. In one alternative, the moiety D can be a moiety containing at least one carboxyl, carboxamide, carboxyl ester, or carbonyl function. In another alternative, the moiety D can be a moiety containing at least one hydroxyl, primary amino, secondary amino, tertiary amino, sulfhydryl, or sulfonamidyl function. The moiety D can be cyclic or acyclic. Preferred examples of the moiety D are described below.

When the moiety D is a cyclic or acyclic moiety containing at least one carbonyl, carboxamide, carboxyl ester, or carbonyl function, in one preferred example, D is a carboxylic acid or carboxylic acid ester with the structure

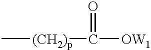

wherein p is an integer from 1 to 6 and W₁ is selected from the group consisting of hydrogen and lower alkyl. Typically, if W₁ is lower alkyl, it is methyl, ethyl, propyl, butyl, or isobutyl. Typically, p is 3. Typically, W₁ is hydrogen or ethyl.

In another preferred example, D and Y₁ are taken together to form a piperazine derivative as described in D. Manetti et al., “Molecular Simplification of 1,4-Diazabicyclo[4.3.0]nonan-9-ones Gives Piperazine Derivatives That Maintain High Nootropic Activity,” J. Med. Chem. 43: 4499-4507 (“Manetti et al. (2000)”). B is an analogue of structure

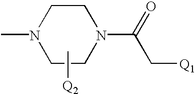

wherein Q₁ is hydrogen, methyl, ethyl, butyl, or propyl, Q₂ is hydrogen or methyl, where, if Q₂ is methyl, it can be located at either of the two possible positions in the piperazine ring.

In another preferred example, D has the structure

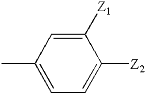

where one of Z₁ and Z₂ is hydrogen, and the other of Z₁ and Z₂ is —COOH or —COOW₁, wherein W₁ is alkyl. Typically, W₁ is selected from the group consisting of methyl, ethyl, propyl, butyl, and isobutyl. Either of Z₁ or Z₂ can be hydrogen. When Z₁ is hydrogen and Z₂ is —COOH, the moiety B is p-aminobenzoic acid (PABA). When Z₁ is —COOH and Z₂ is hydrogen, the moiety B is m-aminobenzoic acid (MABA). When Z₁ is hydrogen and Z₂ is —COOW₁, the moiety B is an ester of p-aminobenzoic acid (PABA). When Z₁ is —COOW₁ and Z₂ is hydrogen, the moiety B is an ester of m-aminobenzoic acid (MABA). Typically, these esters are ethyl esters.

When the moiety D is a moiety that contains at least one hydroxyl, primary amino, secondary amino, tertiary amino, sulfhydryl, or sufonamidyl function, in one preferred example, D is a phenylsulfonamidyl moiety of structure

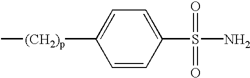

wherein p is an integer from 0 to 6. Typically, p is 2.

In another preferred example, D is an alkylpyridyl moiety of structure

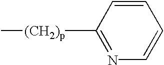

wherein p is an integer from 1 to 6. Typically, p is 1.

In another preferred example, D is a dialkylaminoalkyl moiety of the structure

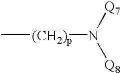

wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y2, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Where Q₇ and Q₈ can be taken together to form a five or six member ring, the ring is typically pyrrolidine, piperidine, or morpholine. The pyrrolidine ring can be optionally substituted with oxo. The piperidine ring can be optionally substituted with methyl or ethyl. Typically, p is 2 or 3.

In another preferred example, D is an alkylpyrrolidine moiety of the structure

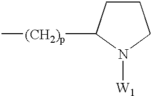

wherein p is an integer from 1 to 6 and W₁ is selected from the group consisting of methyl, ethyl, and propyl. Typically, W₁ is methyl. Typically, p is 2.

Preferably, a compound useful in methods according to the present invention has a log P of from about 1 to about 4 in order to optimize bioavailability and CNS penetration of the compound.

The multidrug transporter protein to be inhibited can be selected from the group consisting of P-glycoprotein and multidrug resistance associated proteins (MRPs).

In methods according to the present invention, the disease or condition to be treated can be a malignancy, a microbial or parasitic infection, HIV infection, or a condition associated with inflammation. Multidrug transporters are found in bacteria and are associated with drug resistance, as described in H. W. van Veen & W. I. Konings, “Multidrug Transporters from Bacteria to Man: Similarities in Structure and Function,” Semin. Cancer Res. 8: 188-191 (1997). The condition associated with inflammation can be asthma or a rheumatic disease.

Another aspect of the present invention is a method of increasing intestinal absorption of a drug transported by a multidrug transporter protein comprising administering to a mammal an effective quantity of a compound as described above.

Yet another aspect of the present invention is a method of improving the penetration of a drug transported by a multidrug transporter into the central nervous system comprising administering to a mammal an effective quantity of a compound as described above.

Yet another aspect of the present invention is a method of decreasing renal excretion or renal toxicity of a drug transported by a multidrug transporter protein comprising administering to a mammal an effective quantity of a compound as described above.

Yet another aspect of the present invention is a method of treating a malignancy comprising:

(a) administering an effective quantity of an antineoplastic agent transported by a multidrug transporter protein to a mammal with cancer; and

(b) administering an effective quantity of a compound as described above.

The antineoplastic agent can be selected from the group consisting of adriamycin, etoposide, vinblastine, actinomycin D, and taxol.

Exemplary studies and treatments were performed as discussed below using various dosages and routes of administration of selected exemplary compounds representative of compositions that are effective with the methods of the present invention. Of course, those skilled in the art will recognize that the present invention is not specifically limited to the particular compositions, dosages or routes of administration detailed below.

Depending upon the particular needs of the individual subject involved, the compositions used in the present invention may be administered in various doses to provide effective treatment concentrations based upon the teachings of the present invention. What constitutes an effective amount of the selected composition will vary based upon such factors including the activity of the selected purine derivative, the physiological characteristics of the subject, the extent and nature of the subject's disease or condition and the method of administration. Exemplary treatment concentrations which have proven effective in modifying neural activity range from less than 1 μM to concentrations of 500 mM or more. Generally, initial doses will be modified to determine the optimum dosage for treatment of the particular mammalian subject. The compositions may be administered using a number of different routes including orally, topically, transdermally, intraperitoneal injection or intravenous injection directly into the bloodstream. Of course, effective amounts of the compounds may also be administered through injection into the cerebrospinal fluid or infusion directly into the brain, if desired.

The methods of the present invention may be effected using compounds administered to a mammalian subject either alone or in combination as a pharmaceutical formulation. Further, the compounds may be combined with pharmaceutically acceptable excipients and carrier materials such as inert solid diluents, aqueous solutions or non-toxic organic solvents. If desired, these pharmaceutical formulations may also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art, including, but not limited to, human serum albumin, ion exchangers, dextrose, alumina, lecithin, buffer substances such as phosphate, glycine, sorbic acid, potassium sorbate, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous, isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. Compositions suitable for use in methods according to the present invention can be administered, for example, by intravenous infusion, orally, topically, intraperitoneally, intravesically, or intrathecally. Formulations of compounds suitable for use in methods according to the present invention can be presented in unit-dose or multi-dose sealed containers, in physical forms such as ampules or vials.

Yet another aspect of the present invention is a screening method to identify compounds capable of inhibiting or modulating the activity of at least one multidrug transporter protein. This method comprises:

(1) adding the compound to a culture of cancer cells that constitutively express or are induced to express at least one multidrug resistance transporter protein;

(2) adding a cytotoxic agent transported by the multidrug resistance transport protein to the cells;

(3) determining the effect of the compound on the activity of the multidrug resistance transporter protein by performing one or both of a cytotoxicity assay and a drug accumulation assay on the cancer cells to measure either the cytotoxicity of the cytotoxic agent or the accumulation of the cytotoxic agent in the cancer cells; and

(4) comparing the effect of the compound on the activity of the multidrug transporter protein with the effect of a reference compound, N-4-carboxyphenyl-3-(6-oxohydropurin-9-yl)propanamide.

The invention is illustrated by the following Example. This Example is presented for illustration only and is not intended to limit the invention.

### EXAMPLE

Involvement of Multidrug Resistance Transporters in Transport of the Bifunctional Purine Derivative N-4-Carboxyphenyl-3-(6-Oxohydropurin-9-yl)Propanamide Out of Brain

AIT-082 (NEOTROFIN™, leteprinim potassium), a hypoxanthine derivative, has robust neurotrophic and neuroprotective actions in vitro and in vivo (reviewed by Rathbone et al., 1999), and is currently under investigation as a possible therapy for humans with mild to moderate Alzheimer's disease (Targum et al., 1999).

Previously, it was demonstrated that AIT-082 is transported into brain by a non-saturable mechanism and, using capillary depletion and microdialysis, it was demonstrated that AIT-082 is detectable in cortical extracellular fluid in low micromolar quantities (Taylor et al., 2000). Additionally, it was demonstrated that, after intracerebroventricular administration, ¹⁴C-AIT-082 was transported out of brain with a t_{½} of approximately 20 minutes. The rate of disappearance from brain was too rapid to be accounted for by passive mechanisms and, indeed, we demonstrated that the efflux of ¹⁴C-AIT-082 from brain was inhibited by excess unlabeled AIT-082 (Taylor et al., 2000). These data suggested that AIT-082 was transported out of brain by a saturable mechanism.

The blood-brain barrier, which creates and maintains the privileged environment of the CNS, comprises three “lines of defense”. The first is a physical barrier formed by tight junctions between endothelial cells of the brain capillaries and epithelial cells of the choroid plexus. Secondly, an enrichment of enzymes including peptidases and drug metabolizing enzymes creates an enzymatic barrier. The third level of protection consists of a collection of transporters that serve to transport compounds from brain to blood. Such transporters include the multidrug transporters, P-glycoprotein (P-gp) and multidrug resistance associated proteins (MRPs) (Banks et al., 1994).

Both P-gp (Cordon-Cardo et al., 1990; Sugawara et al., 1990;Hegmann et al., 1992; Jette et al., 1993; Rao et al., 1999) and MRP (Kusuhara et al., 1998; Regina et al., 1998;Huai-Yun et al., 1998; Gutmann et al., 1999; Nishino et al., 1999; Rao et al., 1999; Seetharaman et al., 1998) are expressed in brain capillary endothelial cells and choroid plexus epithelial cells. The most compelling evidence for the role of the multidrug resistance proteins in blood-brain barrier function has come from gene knockout studies. MDR1a knockout mice, which lack P-gp, show increased blood-brain barrier permeability to digoxin, cyclosporin A, dexamethasone, vinblastine, ondansetron and loperamide and increased sensitivity to the neurotoxic effects of ivermectin (Schinkel et al., 1994, 1995, 1996,1998). Furthermore, P-gp and MRP inhibitors have been shown to enhance the blood-brain barrier penetration of drugs including dideoxyinosine (DDI) (Galinsky et al., 1991), zidovudine (AZT) (Takasawa et al., 1997), cyclosporin A (Didier et al., 1995; Tsujui et al., 1993), quinidine (Kusuhara et al., 1997), colchicine (Drion et al., 1996) and vinblastine (Drion et al., 1996).

The substrate specificity of the P-gp and MRP transporters is broad. P-glycoprotein traditionally transports hydrophobic cationic or neutral compounds (Gottesman et al., 1996), however it has been shown to transport hydrophilic acids such as methotrexate (De Graaf et al., 1996). The MRPs are known to transport organic anions, glutathione conjugates, and peptidyl leukotrienes (Barrand et al., 1997). AIT-082 is a small organic molecule that contains a single aromatic carboxylate anion. These characteristics make AIT-082 a potential substrate for both P-gp and MRP and in this study we have investigated the role of these transporters in the saturable efflux of AIT-082 from brain.

Methods

Animals

Male Swiss-Webster CFW mice were supplied by Charles River Laboratories (Hollister, Calif.) and all experiments were conducted according to the NIH Guide on Care and Use of Laboratory Animals. Mice were 2-3 months old at the time of use.

Materials

AIT-082 (99.5% pure) was synthesized by Eprova (Schaffhausen, Switzerland) and ¹⁴C-AIT-082 (51.5 mCi/mmol; ≧98% pure) was synthesized by Chemsyn Laboratories (Lenexa, Kans., USA). ³H-sucrose (5-15 Ci/mmol) was purchased from Amersham Pharmacia Biotech (Arlington Heights, Ill.) and ³H-quinidine (10-20 Ci/mmol) was from American Radiolabeled Chemicals (St. Louis, Mo.). Probenecid and verapamil hydrochloride were purchased from Sigma Chemical Company (St. Louis, Mo.). MK-571 was purchased from Alexis Biochemicals (San Diego, Calif.).

Intracerebroventricular (icv) Efflux Experiments

These experiments were conducted according to the method of Banks et al. (1997) with minor modifications. The skull was exposed and, with a guarded 25 g needle, a hole was made through the skull at 1 mm anterior posterior (AP) and 1 mm lateral left (LL), relative to Bregma, and 3.5 mm dorsal ventral (DV), with respect to the skull. A guarded 1 μL Hamilton syringe (25 g) was used to inject 1 μL of PBS containing ¹⁴C-AIT-082 (˜4.5×10⁴ dpm/μL), ³H-sucrose (˜3×10⁴ dpm/μL), or ³H-quinidine (˜3×10⁴ dpm/μL) icv into mice. After injection and upon withdrawing the needle, there was often back flux of fluid that was collected. At various times after injection the amount of radioactivity remaining in the brain was determined. The amount of radioactivity in the back flux and injection mixture was also determined. Radioactivity in brain was corrected for the back flux and the log of this corrected dpm was plotted against time. The t_{½}, was the inverse of the slope of the line multiplied by 0.301 (Banks et al., 1997).

To examine the role of P-gp and MRP, various substrates/inhibitors were co-administered with ¹⁴C-AIT-082. The following substrates/inhibitors were used: a) probenecid: 350 mM in PBS containing 370 mM NaOH and 20 mM HCl, pH 7.4; b) verapamil: 200 mM in PBS containing 4.2% ethanol, pH 7.4; c) MK-571: 1,10 or 100 mM in PBS. In all experiments the control group of animals was given ¹⁴C-AIT-082 in PBS. Additionally, in experiments in which probenecid or verapamil were used, a second control group was given ¹⁴C-AIT-082 in PBS containing 370 mM NaOH and 370 mM HCl, pH 7.4, or 14C-AIT-082 in PBS with 4.2% ethanol, pH 7.4, respectively.

Intraparenchymal (ipc) Efflux Experiments

These experiments were conducted according the method of Banks et al. (1994) with minor modifications. Mice were anesthetized and then immobilized in a stereotaxic apparatus with a mouse adaptor coupled to a microinjection unit (Kopf, Tujunga, Calif.). A small hole was made in the skull with a Dremel drill (model 770; 2.4 mm drill bit, model 107) at −1 mm AP and 1 mm LL relative to Bregma. Using a guarded 0.5 μL Hamilton syringe (25 g), 0.1 μL PBS containing ¹⁴C-AIT-082 (˜5×10 dpm/μL) or ³H-sucrose (˜5×10⁴ dpm/μL) was injected ipc at 3.5 min DV with respect to the skull. Back flux of injection fluid was collected and at various times after injection brains were removed. The amount of radioactivity in brain, back flux and injection mixture was measured. The amount of radioactivity in the brain was corrected for the back flux and the log of this corrected dpm was plotted against time. The t_{½} was calculated as described above.

P-gp and MRP substrates/inhibitors were co-administered with ¹⁴C-AIT-082 in the following concentrations: a) verapamil: 2 mM in water with less than 0.05% ethanol or 200 mM in water, 4% ethanol and 20 mM NaOH; b) MK-571: 10 mM in PBS. As above, all experiments included a control group in which ¹⁴C-AIT-082 was administered in PBS. In the experiments in which verapamil was used, a second control group received ¹⁴C-AIT-082 in water containing 4% ethanol and 20 mM NaOH.

Statistical Analysis

All data are presented as mean ±S.E. For comparisons of means from 2 groups an unpaired Student's t-test was used. For comparisons of means from 3 groups, ANOVA was performed coupled with Scheffe's post-hoc analysis.

Results

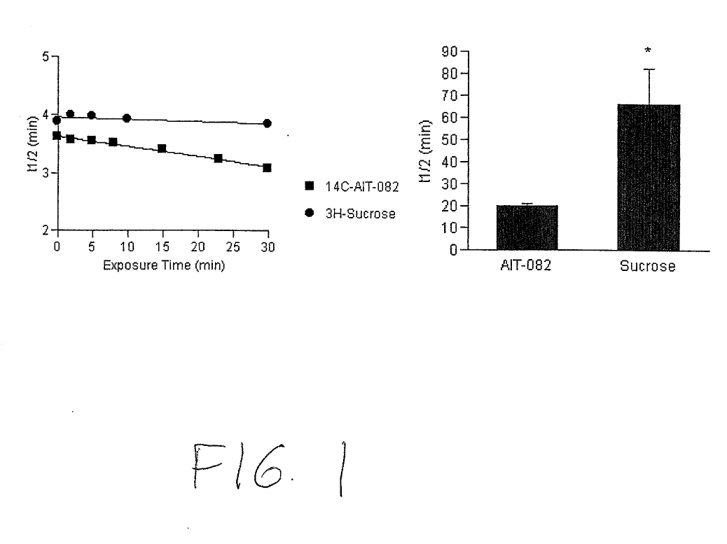

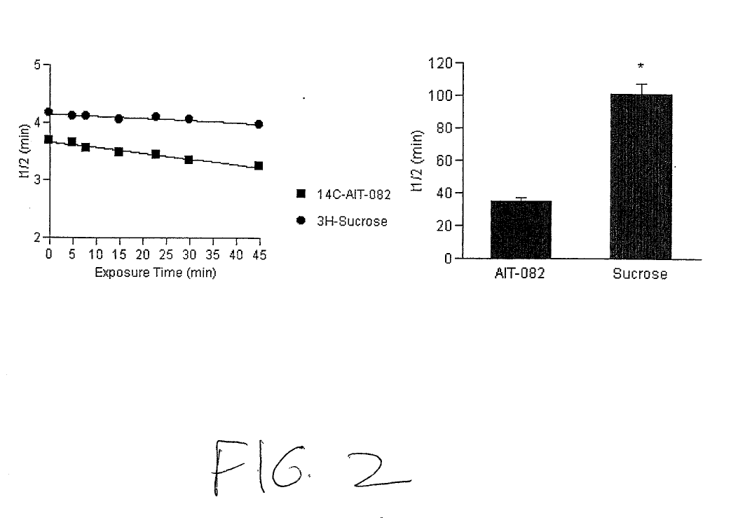

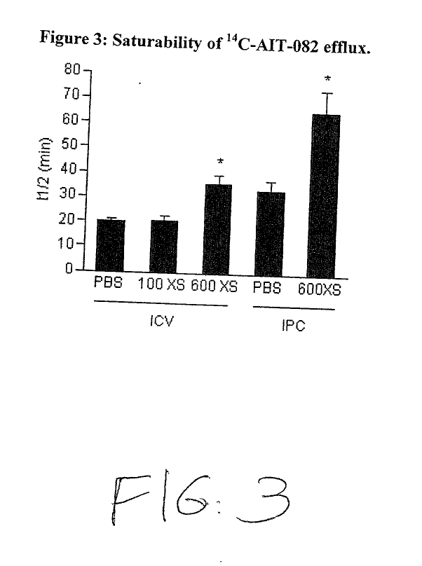

After both icv (FIG. 1) and ipc (FIG. 2) administration, ¹⁴C-AIT-082 was transported out of brain in an exponential manner with a t_{½} of 20±1.0 and 35.0±1.9 minutes, respectively. In both cases transport of ³H-sucrose, a passively transported compound, was significantly slower (i.e. the t_{½} was significantly higher). Administration of ¹⁴C-AIT-082 icv exposes the compound to both the capillary endothelial cells and the choroid plexus epithelial cells of the blood-brain barrier. In contrast, when compounds are administered ipc, the endothelial cells are effectively isolated and efflux across this barrier can be examined. Thus, these data suggest that AIT-082 is transported out of brain by a saturable mechanism likely located at both the choroid plexus and brain capillary endothelium. This hypothesis was confirmed by the demonstration that excess unlabeled AIT-082 inhibited the efflux of ¹⁴C-AIT-082 from brain after icv and ipc administration (FIG. 3).

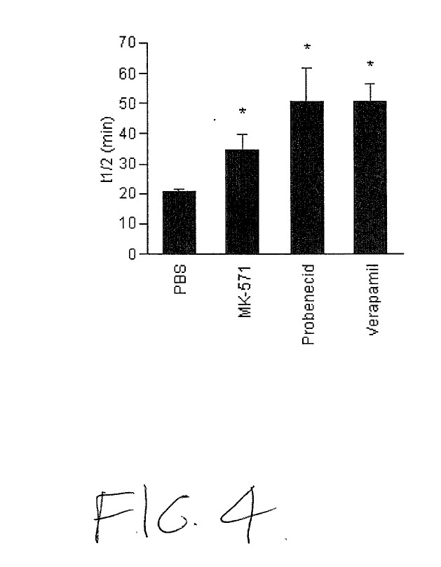

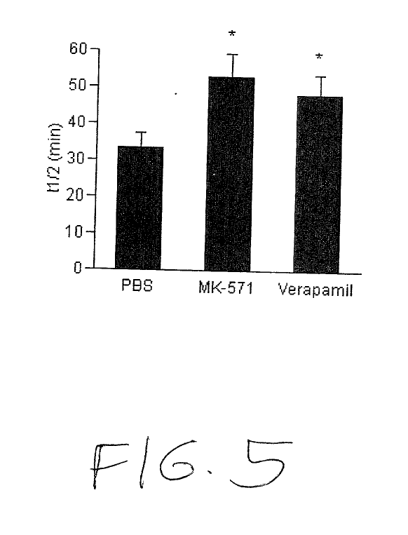

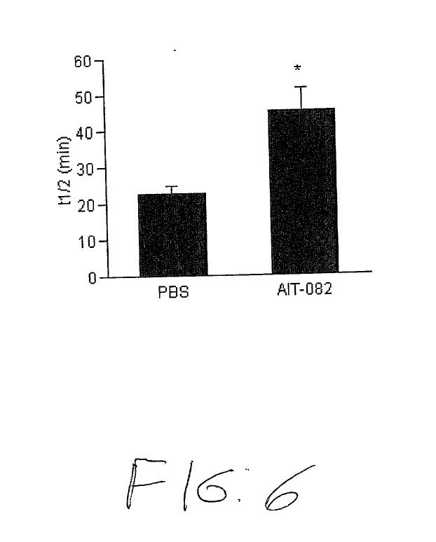

The role of P-gp and MRP in the transport of ¹⁴C-AIT-082 out of brain was investigated. Verapamil, an inhibitor of P-gp, and both probenecid and MK-571, inhibitors of MRPs, significantly inhibited efflux of ¹⁴C-AIT-082 after icv (FIG. 4) and ipc administration (FIG. 5). In addition, AIT-082 inhibited the efflux of ³H-quinidine, a P-gp substrate, from brain after icv administration (FIG. 6).

Conclusions:

In conclusion, the data presented demonstrate that AIT-082 is transported out of brain by a saturable mechanism that is likely localized to both brain capillary endothelium and epithelium of the choroid plexus. The data indicate that P-gp and MRPs may mediate this efflux.

### REFERENCES

The following references are provided for the Example:

Banks W A, Fasold M B and Kastin A J (1997) Measurement of efflux rates from brain to blood, in Methods in Molecular Biology, Neuropeptide Protocols (Irvine G B and Williams C H eds) pp 353-360,Humana Press Inc., Totowa, N.J.

Banks W A, Kastin A J, Sam H M, Cao V T, King B, Maness L M and Schally A V (1994) Saturable efflux of the peptides RC-160 and Tyr-MIF-1 by different parts of the blood-brain barrier. Brain Res. Bull. 35: 179-182.

Barrand M A, Bagrij T and Neo S-Y (1997) Multidrug resistance-associated protein: A protein distinct from P-glycoprotein involved in cytotoxic drug expulsion. Gen. Pharmac. 28: 639-645.

Cordon-Cardo C, O'Brien J P, Boccia J, Casals D, Bertino J R and Melamed M R (1990) expression of the multidrug resistance gene product (P-glycoprotein) in human normal and tumor tissues. J. Histochem. Cytochem. 38: 1277-1287.

De Graaf D, Sharma R C, Mechetner E B, Schimke R T and Roninson I B (1996) Pglycoprotein confers methotrexate resistance in 3T6 cells with deficient carrier-mediated methotrexate uptake. Proc. Natl. Acad. Sci. USA 93: 11238-1242.

Didier A D and Loor F (1995) Decreased biotolerability for ivermectin and cyclosporin A in mice exposed to potent P-glycoprotein inhibitors. Int.J. Cancer 63: 263-267.

Drion N, Lemaire M, Lefauconnier J-M and Schermann J-M (1996) Role of P-glycoprotein in the blood-brain transport of colchicine and vinblastine J. Neurochem. 67: 1688-1693.

Galinsky R E, Flaharty K K, Hoesterey B L and Anderson B D (1991) Probenecid enhances central nervous system uptake of 2′,3′-dideoxyinosine by inhibiting cerebrospinal fluid efflux. J. Pharmacol. Exp. Therap. 257: 972-978.

Gottesman M M, Pastan I and Ambudkar S V (1996) P-glycoprotein and multidrug resistance. Curr. Opin. Genet Dev. 6: 610-617.

Gutmann H, Torok M, Fricker G, Huwyler J, Beglinger C and Drewe J (1999) Modulation of multidrug resistance protein expression in porcine brain capillary endothelial cells in vitro. Drug Metab. Disp. 27: 937-941.

Hegmann. E J, Bauer H C and Kerbel R S (1992) Expression and functional activity of P-glycoprotein in cultured cerebral capillary endothelial cells. Cancer Res. 52: 6969-6975.

Huai-Yun H, Secrest D I, Mark K S, Carney D, Brandquist C, Elmquist W F and Miller D W (1998) Expression of multidrug resistance-associated protein (MRP) in brain microvessel endothelial cells. Biochem. Biophys. Acta 243: 816-820.

Jette L, Tetu B and Beliveau R (1993) high levels of P-glycoprotein detected in isolated brain capillaries. Biochem. Bophys. Acta 1150: 147-154.

Kusuhara H, Suzuki H, Terasaki T, Kakee A, Lemaire M and Sugiyama Y (1997) P-glycoprotein mediates the efflux of quinidine across the blood-brain barrier. J. Pharm. Exp. Therap. 283: 574-580.

Kusuhara H, Suzuki H, Naito M, Tsuruo T and Sugiyama Y (1998) Characterization of efflux transport of organic anions in a mouse brain capillary endothelial cell line. J. Pharm, Exp. Therap. 285:1260-1265.

Nishino J-I, Suzuki H, Sugiyama D, Kitazawa T, Ito K, Hanano M and Sugiyama Y (1999) Transepithelial transport of organic anions across the choroid plexus: possible involvement of organic anion transporter and multidrug resistance-associated protein. J. Pharm. Exp. Therap. 290: 289-294.

Rao V V Dahlheimer J L, Bardgett M E, Snyder A Z, Finch R A, Sartorelli A C and Piwnica-Worms D (1999) Choroid plexus epithelial expression of MDR1 P glycoprotein and multidrug resistance-associated protein contribute to the blood-cerebrospinal fluid drug-permeability barrier. Proc. Natl. Acad. Sci. USA 96: 3900-3905.

Rathbone M P, Middlemiss P J, Crocker C E, Glasky M S, Juurlink B H J, Ramirez J J, Ciccarelli R, Dilorio P, and Caciagli F (1999) AIT-082 as a potential neuroprotective and regenerative agent in stroke and central nervous system injury. Exp. Opin. Invest Drugs 8: 1255-1262.

Regina A, Koman A, Piciotti M, El Hafny B, Center MS, Bergmann R, Couraud P and Roux F (1998) Mrp1 multidrug resistance-associated protein and P-glycoprotein expression in rat brain microvessel endothelial cells. J. Neurochem. 71: 705-715.

Schinkel A H, Smit J J M, van Tellingen O, Beijnen J H, Wagenaar E, van Deemter L, Mol C M, van der Valk M A, Robanus-Maandag E C M, te Riele H P J, Berns A J M and Borst P (1994) Disruption of the mouse mdr1a P-glycoprotein gene leads to a deficiency in the blood-brain barrier and to increased sensitivity to drugs. Cell 77: 491-502.

Schinkel A H, Wagenaar E, van Demmter L, Mol C M and Borst, P (1995) Absence of the mdr1a P-glycoprotein in mice affects tissue distribution and pharmacokinetics of dexamethasone, digoxin, and cyclosporin A. J. Clin. Invest. 96: 1698-1705.

Schinkel A H, Wagenaar E, Mol C A A and van Deemter L (1996) P-glycoprotein in the blood-brain barrier of mice influences the brain penetration and pharmacological activity of many drugs. J. Clin. Invest 97: 2517-2524.

Schinkel A H (1998) Pharmacological insights from P-glycoprotein knockout mice. Int. J Clin. Pharm. Therap. 36: 9-13.

Seetharaman S, Barrand M A, Maskell L and Scheper R J (1998) Multidrug resistance-related transport proteins in isolated human brain microvessels and in cells cultured from these isolates. J. Neurochem. 70: 1151-1159.

Takasawa K, Terasaki T, Suzuki H and Sugiyama Y (1997) In vivo evidence for carrier-mediated efflux transport of 3′-azo-3′-deoxythymidine and 2′,3′-dideoxyinosine across the blood-brain barrier via a probenecid-sensitive transport system. J. Pharmacol. Exp. Therap. 281: 369-375.

Targum S D, Wieland D S, Glasky M S and Glasky A J (1999) Evaluation of AIT-082 in patients with mild to moderate senile dementia of the Alzheimer's type. European Congress of Neuropsychopharmacology September 21-24, London, UK.

Taylor E M, Yan R, Hauptmann N, Maher T J, Djahandideh D and Glasky A J (2000) AIT-082, a cognitive enhancer, is transported into brain by a non-saturable influx mechanism and out of brain by a saturable efflux mechanism. J. Pharmacol. Exp. Therap. 293: 813-821.

Tsuji A, Tamai I, Sakata A, Tenda Y and Terasaki T (1993) Restricted transport of cyclosporin A across the blood-brain barrier by a multidrug transporter, P-glycoprotein. Biochem Pharmacol. 46: 1096-1099.

Advantages of the Invention

The present invention provides new methods for treating diseases and conditions affected by activity of a multidrug transporter protein by inhibiting or modulating the activity of such a protein. These methods can be combined with methods that enable active compounds to bypass the blood-brain barrier, making combined therapy more efficient. These methods are suitable for use with a large variety of active compounds and should not depend on the specific interactions between each active compound and the transporter proteins. The methods of the invention are useful in treating malignancies, microbial and parasitic infections, HIV infection, and conditions associated with inflammation, such as asthma and rheumatic disease. The invention provides particular advantages in treating malignancies, as it allows the use of smaller doses of potentially toxic anticancer agents, thus reducing the likelihood of side effects such as immune suppression.

Although the present invention has been described in considerable detail, with reference to certain preferred versions thereof, other versions and embodiments are possible. Therefore, the scope of the invention is determined by the following claims.

We claim:

1. A method of treating a disease or condition associated with the activity of a multi-drug transporter protein comprising administering to a patient suffering from a condition or disease associated with the activity of a multi-drug transporter protein an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L though a carbonyl group wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

2. The method of claim 1 wherein the compound having activity against a multi-drug transporter protein passes through the blood-brain barrier.

3. The method of claim 1 wherein A is a purine moiety.

4. The method of claim 3 wherein A is a substituted or unsubstituted hypoxanthine moiety.

5. The method of claim 4 wherein L has the structure —(CH₂)ₙ—CONH— where n is an integer from 1 to 6.

6. The method of claim 5 wherein the compound having activity against a multi-drug transporter protein is a compound of formula (I) 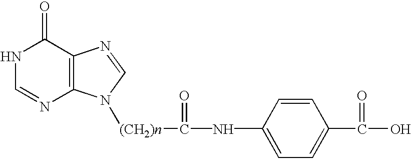
where n is an integer from 1 to 6 and R is hydrogen or lower alkyl or is a salt or prodrug ester of a compound of formula (I) 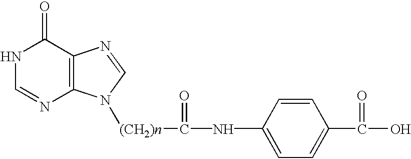
wherein n is an integer from 1 to 6 and R is hydrogen or lower alkyl.

7. The method of claim 6 wherein the compound having activity against a multi-drug transporter protein is a compound of formula (I) wherein n is an integer from 1 to 6 and R is hydrogen or lower alkyl.

8. The method of claim 7 wherein R is hydrogen.

9. The method of claim 8 wherein n is 2 and the compound is N-4-[[3-(1,6-dihydro-6-oxo-purin-9-yl)-1-oxopropyl]amino]benzoic acid.

10. The method of claim 7 wherein R is ethyl.

11. The method of claim 10 wherein n is 2 and the compound is N-4-[[3-(1,6-dihydro-6-oxo-purin-9-yl)-1-oxopropyl]amino]benzoic acid ethyl ester.

12. The method of claim 5 wherein the compound having activity against a multi-drug transporter protein is a compound of formula (II) 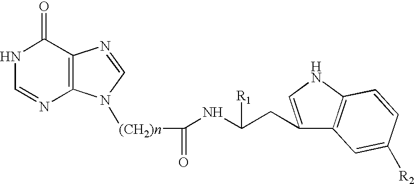
wherein n is an integer from 1 to 6, R is selected from the group consisting of H, COOH, and COOW₁, wherein W₁ is selected from the group consisting of lower alkyl, amino, and lower alkylamino, and R₂ is selected from the group consisting of H and OH.

13. The method of claim 12 wherein n is 2.

14. The method of claim 5 wherein the compound having activity against a multi-drug transporter protein is a compound of formula (III) 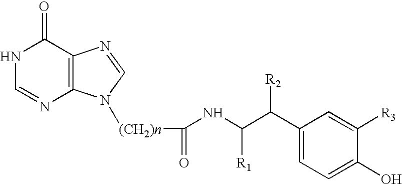
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting from the group consisting of H and OH.

15. The method of claim 14 wherein n is 2.

16. The method of claim 3 wherein A is a substituted or unsubstituted guanine moiety.

17. The method of claim 16 wherein L has the structure —(CH₂)ₙ—CONH— wherein n is an integer from 1 to 6.

18. The method of claim 17 wherein the compound having activity against a multi-drug transporter protein is a compound of formula (IV) 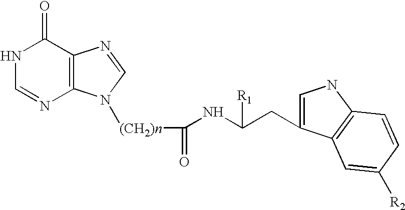
wherein n is an integer from 1 to 6, R₁ is selected from the group consisting of H, COOH, and COOW₁, wherein W₁ is selected from the group consisting of lower alkyl, amino, and lower alkylamino and R₂ is selected from the group consisting of H and OH.

19. The method of claim 18 wherein n is 2, R₁ is H, and R₂ is OH, and the compound is N—(2-(5-hydroxyindol-3-yl)) ethyl-3-(2-amino-6-oxohydropurin-9-yl)propanamide.

20. The method of claim 18 wherein n is 2, R₁ is H, and R₂ is H, and the compound is N—(2-(2-indol-3-yl)ethyl))-3-(2-amino-6-oxohydropurin-9-yl)propanamide.

21. The method of claim 18 wherein n is 2, R₁ is COOH, and R₂ is OH, and the compound is N—(1-carboxyl-(2-(5-hydroxyindol-3-yl)ethyl)-3-(2-amino-6-oxohydropurin-9-yl)propanamide.

22. The method of claim 17 wherein the compound having activity against a multi-drug transporter protein is a compound of formula (V) 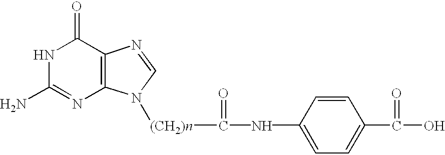
wherein n is an integer from 1 to 6 and R is selected from the group consisting of hydrogen and lower alkyl.

23. The method of claim 22 wherein n is 2, R is hydrogen, and the compound is N-4-carboxyphenyl-3-(2-amino-6-oxohydropurin-9-yl)propanamide.

24. The method of claim 22 wherein n is 2, R is ethyl, and the compound is N-4-carboxyphenyl-3-(2-amino-6-oxohydropurin-9-yl)propanamide ethyl ester.

25. The method of claim 17 wherein the compound having activity against a multi-drug transporter protein is a compound of formula (VI) 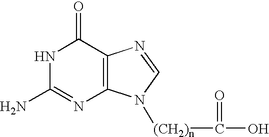
wherein n is an integer from 1 to 6 and R is selected from the group consisting of hydrogen and lower alkyl.

26. The method of claim 25 wherein n is 2, R is hydrogen, and the compound is 3-(2-amino-6-oxohydropurin-9-yl)propanoic acid.

27. The method of claim 25 wherein n is 2, R is ethyl, and the compound is 3-(2-amino-6-oxohydropurin-9-yl)propanoic acid ethyl ester.

28. The method of claim 17 wherein the compound having activity against a multi-drug transporter protein is a compound of formula (VII) 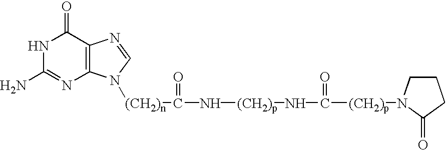
wherein n is an integer from 1 to 6, p is an integer from 1 to 6, and q is an integer from 1 to 3.

29. The method of claim 28 wherein n is 2, p is 2, and q is 1, and the compound is N-[2-[[2-(2-oxopyrrolidin-1-yl)-1-oxoethyl]amino]ethyl]propanamide.

30. The method of claim 17 wherein the compound having activity against a multi-drug transporter protein is a compound of formula (VIII) 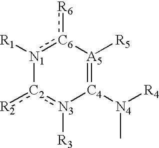
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

31. The method of claim 30 wherein n is 2, R₁ is H, R₂ is H, and R₃ is OH, and the compound is N—(2-(3,4-dihydroxyphenyl)ethyl-3-(2-amino-6-oxohydropurin-9-yl)propanamide.

32. The method of claim 30 wherein n is 2, R₁ is H, R₂ is OH, and R₃ is OH, and the compound is N—(2-hydroxy-2-(3,4-dihydroxyphenyl)ethyl)-3-(2-amino-6-oxohydropurin-9-yl)propanamide.

33. The method of claim 30 wherein n is 2, R₁ is COOH, R₂ is H, and R₃ is H, and the compound is N—(1-carboxyl-2-(3,4-dihydroxyphenyl)ethyl)-3-(2-amino-6-oxohydropurin-9-yl)propanamide.

34. The method of claim 16 wherein the compound having activity against a multi-drug transporter protein is a compound of formula (IX) 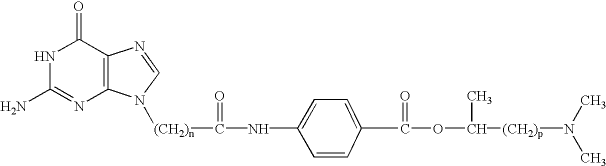
wherein n is an integer from 1 to 6 and p is an integer from 1 to 3.

35. The method of claim 34 wherein n is 2, p is 1, and the compound is N-4-[[3-(2-amino-6-oxohydropurin-9-yl) 1-oxopropyl]amino]benzoic acid 1-(dimethylamino)-2-propyl ester.

36. The method of claim 1 wherein A is a substituted or unsubstituted 9-atom bicyclic moiety in which the 5-membered ring has 1 to 3 nitrogen atoms, the bicyclic moiety having the structure of formula (X) 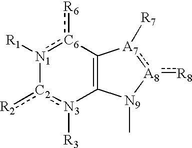
where:
(a) if the bond between N₁ and the bond between C5 is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C2 and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) A₇ and A₈ are C or N;
(i) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;
(ii) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;
(iii) if A₇ and A₈ are both N, then the bond between A₇ and A₈ is a double bond, and R₇ and R₈ are not present;
(iv) if A₇ is C and A₈ is N, then the bond between A₇ and A₈ is a double bond, R₇ is hydrogen, and R₈ is not present;
(v) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;
(vi) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and
(f) N₉ is bonded to L; with the proviso that A does not have the structure of an unsubstituted guanine or hypoxanthine.

37. The method of claim 3 wherein the purine moiety is a purine moiety of formula (XI) 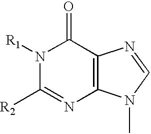
in which:
(a) R₁ is selected from the group consisting of hydrogen, alkyl, aralkyl, cycloalkyl, and heteroaralkyl; and R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

38. The method of claim 37 wherein R₁ is butyl and R₂ is hydrogen.

39. The method of claim 37 wherein R₁ is benzyl and R₂ is hydrogen.

40. The method of claim 37 wherein R₁ is dimethylaminoethyl and R₂ is hydrogen.

41. The method of claim 37 wherein R₁ is cyclopentyl and R₂ is hydrogen.

42. The method of claim 37 wherein R₁ is cyclohexylmethyl and R₂ is hydrogen.

43. The method of claim 37 wherein R₁ is cyclopropylmethyl and R₂ is hydrogen.

44. The method of claim 37 wherein R₁ is hydrogen and R₂ is phenyl.

45. The method of claim 37 wherein R₁ is hydrogen and R₂ is butyl.

46. The method of claim 37 wherein R₁ is butyl and R₂ is butyl.

47. The method of claim 37 wherein R₁ is hydrogen and R₂ is methyl.

48. The method of claim 37 wherein R₁ is hydrogen and R₂ is phenylamino.

49. The method of claim 3 wherein the purine moiety is a purine moiety of Formula (XII) 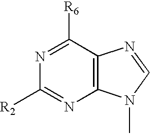
in which:
(a) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(b) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

50. The method of claim 49 wherein R₂ is hydrogen and R₆ is amino.

51. The method of claim 49 wherein R₆ is chloro.

52. The method of claim 49 wherein R₆ is phenylamino.

53. The method of claim 49 wherein R₂ is amino and R₆ is chloro.

54. The method of claim 3 wherein the purine moiety is a purine moiety of Formula (XIII) 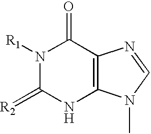
in which:
(a) R₁ is hydrogen, alkyl, aralkyl, cycloalkyl, or heteroaralkyl; and
(b) R₂ is O or S.

55. The method of claim 54 wherein R₁ is hydrogen.

56. The method of claim 54 wherein R₂ is O.

57. The method of claim 54 wherein R₂ is S.

58. The method of claim 3 wherein the compound is 4-[3-(1-benzyl-6-oxo-1,6-dihydropurin-9-yl)propionylamino]benzoic acid ethyl ester.

59. The method of claim 3 wherein the compound is 4-[3-(1-butyl-6-oxo-1,6-dihydropurin-9-yl)propionylamino]benzoic acid ethyl ester.

60. The method of claim 3 wherein the compound is 4-[3-(1-methyl-6-oxo-1,6-dihydropurin-9-yl)propionylamino]benzoic acid ethyl ester.

61 The method of claim 3 wherein the compound is 4-[3-(1-2-dimethylaminoethyl)-6-oxo-1,6-dihydropurin-9-yl)propionylamino]benzoic acid ethyl ester.

62. The method of claim 3 wherein the compound is 4-[3-(2,6-dioxo-1,2,3,6-tetrahydropurin-9-yl)propionylamino]benzoic acid ethyl ester.

63. The method of claim 3 wherein the compound is 4-[3-(6-methoxypurin-9-yl)propionylamino]benzoic acid ethyl ester.

64. The method of claim 3 wherein the compound is 4-[3-(6-dimethylaminopurin-9-yl)propionylamino]benzoic acid ethyl ester.

65. The method of claim 3 wherein the compound is 4-[3-(2-amino-6-chloropurin-9-yl)propionylamino]benzoic acid ethyl ester.

66. The method of claim 3 wherein the compound is 4-[2-(6-oxo-2-thioxo-1,2,3,6-tetrahydropurin-9-yl)propionylamino]benzoic acid ethyl ester.

67. The method of claim 3 wherein the compound is 4-[2-(2-butyl-6-oxo-1,6-dihydropurin-9-yl)propionylamino]benzoic acid ethyl ester.

68. The method of claim 3 wherein the compound is 4-[2-(6-oxo-2-phenyl-1,6-dihydropurin-9-yl)propionylamino]benzoic acid ethyl ester.

69. The method of claim 3 wherein the compound is 4-{[3-(6-chloropurin-9-yl) propionyl]methylamino}benzoic acid methyl ester.

70. The method of claim 3 wherein the compound is 3-(1-benzyl-6-oxo-1,6-dihydropurin-9-yl)-N-[3-(2-oxopyrrolidin-1 -yl)propyl]propanamide.

71. The method of claim 3 wherein the compound is 3-(1-benzyl-6-oxo-1,6-dihydropurin-9-yl)-N-{2-[2-(2-oxopyrrolidin-1-yl)acetylamino]ethyl}propanamide.

72. The method of claim 3 wherein the compound is N-[3-(2-oxopyrrolidin-1-yl)propyl]-3-(6-oxo-2-thioxo-1,2,3,6-tetrahydropurin-9-yl)propanamide.

73. The method of claim 3 wherein the compound is 3-(1-benzyl-6-oxo-1,6-dihydropurin-9-yl)-N-(3-morpholin-4-yl)propyl propionamide.

74. The method of claim 1 wherein the compound is a tetrahydroindolone derivative or analogue where A is a 9-atom bicyclic moiety in which the 5-membered ring has one to three nitrogen atoms, the bicyclic moiety of Formula (XIV) 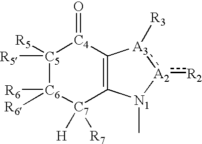
where:
(a) N₁ is bonded to L;
(b) A₂ and A₃ are C or N;
(i) If A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;
(ii) If A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;
(iii) If A₂ and A₃ are both N, then the bond between A₂ and A₃ is a double bond and R₂ and R₃ are not present;
(iv) If A₂ is N and A₃ is C, then the bond between A₂ and A₃ is a double bond, R₂ is not present, and R₃ is hydrogen;
(v) If A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;
(vi) If A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;
(c) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(d) R_{5′} is hydrogen unless R₅ is alkyl, in which case R₅ is hydrogen or the same alkyl as R₅;
(e) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;
(f) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(g) R_{6′} is hydrogen unless R₆ is alkyl, in which case R_{6′} is hydrogen or the same alkyl as R₆;
(h) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S; and
(i) R₇ is hydrogen unless R₅ is alkyl and R_{5′} is hydrogen, in which case R₇ is the same alkyl as R₅.

75. The method of claim 74 wherein A is a tetrahydroindolone moiety.

76. The method of claim 75 wherein the tetrahydroindolone moiety is a tetrahydroindolone moiety of formula (XV) 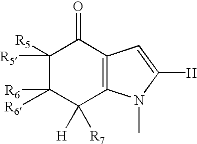
in which:
(a) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;
(b) R_{5′} is hydrogen;
(c) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;
(d) R is hydrogen; and
(e) R₇ is hydrogen.

77. The method of claim 76 wherein R₅, R_{5′}, R₆, R_{6′}, and R₇ are all hydrogen.

78. The method of claim 77 wherein the compound is 4-[3-(4-oxo-4,5,6,7-tetrahydroindolon-1-yl)propionylamino]benzoic acid ethyl ester.

79. The method of claim 77 wherein the compound is 4-[3-(4-oxo-4,5,6,7-tetrahydroindolon-1-yl)propionylamino]benzoic acid.

80. The method of claim 1 wherein A is an amino-substituted 6-membered heterocyclic moiety of formula (XVI) 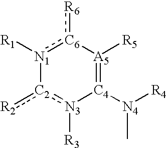
where:
(a) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C2 and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;
(f) A₅ is carbon or nitrogen;
(g) if A₅ is nitrogen, then R₅ is not present;
(h) if A₅ is carbon, then R₅ is hydrogen, amino, alkyl, alkoxy, halo, nitro, aryl, cyano, alkenyl, or alkaryl;
(i) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(j) N₄ is bonded to L.

81. The method of claim 80 wherein A₅ is carbon and the 6-membered heterocyclic moiety is a pyrimidine moiety.

82. The method of claim 81 wherein R₂ is 0 and R₃ is hydrogen.

83. The method of claim 82 wherein the pyrimidine moiety is selected from the group consisting of cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, and 5-hydroxymethyluracil.

84. The method of claim 81 wherein R₂ is S and R₃ is hydrogen.

85. The method of claim 84 wherein the pyrimidine moiety is selected from the group consisting of 2-thiouracil, 5-methylamino-2-thiouracil, 5-methyl-2-thiouracil, 2-thiocytosine.

86. The method of claim 81 wherein R₂ is amino and the bond between C₂ and N₃ is a double bond.

87. The method of claim 86 wherein the pyrimidine moiety is selected from the group consisting of 2-aminopyrimidinone and 2-amino-4-chloropyrimidine.

88. The method of claim 81 wherein R₂ is hydrogen and the bond between C₂ and N₃ is a double bond.

89. The method of claim 88 wherein the pyrimidine moiety is selected from the group consisting of 4-chloropyrimidine, 5-amino-4-chloropyrimidine, 4-chloro-5-methylpyrimidine, 4-chloro-5-hydroxymethylpyrimidine, and 4-chloro-5-carboxymethylpyrimidine.

90. The method of claim 81 wherein R₁ is hydrogen, methyl, or ethyl, R₅ is hydrogen, methyl, or ethyl, and R₆ is O.

91. The method of claim 90 wherein the pyrimidine moiety is pyrimidinone.

92. The method of claim 81 wherein the compound is 4-[3-(2-amino-6-chloropyrimidin-4-ylamino)propionylamino]benzoic acid ethyl ester.

93. The method of claim 81 wherein the compound is 4-[3-(5-amino-6-chloropyrimidin-4-ylamino)propionylamino]benzoic acid ethyl ester.

94. The method of claim 81 wherein the compound is 4-[3-(6-chloropyrimidin-4-ylamino)propionylamino]benzoic acid ethyl ester.

95. The method of claim 81 wherein the compound is 4-[3-(2-amino-6-chloropyrimidin-4-ylamino)propionylamino]benzoic acid.

96. The method of claim 81 wherein the compound is 4-[3-(6-chloropyrimidin-4-ylamino)propionylamino]benzoic acid.

97. The method of claim 81 wherein the compound is 4-[3-(5-amino-6-chloropyrimidin-4-ylamino)propionylamino]benzoic acid.

98. The method of claim 81 wherein the compound is 3-[3-(2-amino-6-chloropyrimidin-4-ylamino)propionylamino]benzoic acid ethyl ester.

99. The method of claim 81 wherein the compound is 3-[3-(6-chloropyrimidin-4-ylamino)propionylamino]benzoic acid ethyl ester.

100. The method of claim 81 wherein the compound is 3-[3-(5-amino-6-chloropyrimidin-4-ylamino)propionylamino]benzoic acid ethyl ester.

101. The method of claim 81 wherein the compound is 3-[3-(2-amino-6-chloropyrimidin-4-ylamino)propionylamino]benzoic acid.

102. The method of claim 81 wherein the compound is 3-[3-(6-chloropyrimidin-4-ylamino)propionylamino]benzoic acid.

103. The method of claim 81 wherein the compound is 3-[3-(5-amino-6-chloropyrimidin-4-ylamino)propionylamino]benzoic acid.

104. The method of claim 1 wherein L has the structure —(CH₂)ₙ— wherein n is an integer from 1 to 6.

105. The method of claim 104 wherein n is 2.

106. The method of claim 104 wherein n is 3.

107. The method of claim 1 wherein the moiety B is —OZ.

108. The method of claim 107 wherein Z is hydrogen.

109. The method of claim 107 wherein Z is alkyl.

110. The method of claim 109 wherein Z is selected from the group consisting of methyl, ethyl, butyl, propyl, and isopropyl.

111. The method of claim 1 wherein B is —N(Y₁)-D.

112. The method of claim 111 wherein Y₁ is hydrogen.

113. The method of claim 111 wherein Y₁ is lower alkyl.

114. The method of claim 113 wherein Y₁ is methyl.

115. The method of claim 111 wherein D is a moiety having at least one polar, charged, or hydrogen-bond-forming group to increase the water-solubility of the compound.

116. The method of claim 115 wherein D is a carboxylic acid or carboxylic acid ester with the structure 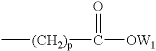
wherein p is an integer from 1 to 6 and W₁ is selected from the group consisting of hydrogen and lower alkyl.

117. The method of claim 116 wherein W₁ is hydrogen.

118. The method of claim 116 wherein W₁ is ethyl.

119. The method of claim 115 wherein D and Y₁ are taken together to form a piperazine derivative of the structure 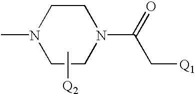
wherein Q₁ is hydrogen, methyl, ethyl, butyl, or propyl, and Q₂ is hydrogen or methyl, where, if Q₂ is methyl, it can be located on either of the two possible positions in the piperazine ring.

120. The method of claim 115 wherein D has the structure 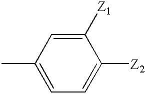
wherein one of Z₁ and Z₂ is hydrogen and the other is Z₁ and Z₂ is —COOH or —COOW₁, wherein W₁ is alkyl.

121. The method of claim 120 wherein W₁ is selected from the group consisting of methyl, ethyl, propyl, butyl, and isobutyl.

122. The method of claim 115 wherein D is a phenylsulfonamidyl moiety of the structure 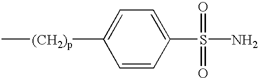
wherein p is an integer from 0 to 6.

123. The method of claim 115 wherein D is an alkylpyridyl moiety of the structure 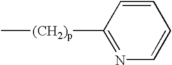
wherein p is an integer from 1 to 6.

124. The method of claim 114 wherein D is an dialkylaminoalkyl moiety of the structure 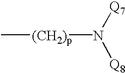
wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S, and when Q₇ and Q₈ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

125. The method of claim 124 wherein Q₇ and Q₈ are each alkyl.

126. The method of claim 125 wherein Q₇ and Q₈ are each selected from the group consisting of methyl, ethyl, propyl, butyl, and isobutyl.

127. The method of claim 126 wherein Q₇ and Q₈ are taken together to form 5-or 6-membered optionally substituted ring.

128. The method of claim 127 wherein the ring is a morpholinyl ring.

129. The method of claim 127 wherein the ring is a pyrrolidinyl ring that is optionally substituted with oxo.

130. The method of claim 126 wherein the ring is a piperidinyl ring that is optionally substituted with methyl or ethyl.

131. The method of claim 115 wherein D is an alkylpyrrolidinyl moiety of the structure 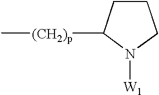
wherein p is an integer from 1 to 6 and W₁ is selected from the group consisting of methyl, ethyl, and propyl.

132. The method of claim 1 wherein the compound has a log P of from about 1 to about 4.

133. The method of claim 1 wherein the multidrug transporter protein is selected from the group consisting of P-glycoprotein and multidrug resistance associated proteins (MRPs).

134. The method of claim 133 wherein the multidrug transporter protein is P-glycoprotein.

135. The method of claim 133 wherein the multidrug transporter protein is MRP.

136 The method of claim 1 wherein the condition or disease associated with the activity of a multidrug transporter protein is selected from the group consisting of cancer, a microbial or parasitic infection, HIV infection, and a condition associated with inflammation.

137. The method of claim 136 wherein the condition or disease is cancer.

138. The method of claim 136 wherein the condition or disease is a microbial or parasitic infection.

139. The method of claim 136 wherein the condition or disease is a HIV infection.

140 The method of claim 136 wherein the condition or disease is a condition associated with inflammation.

141. The method of claim 140 wherein the condition associated with inflammation is selected from the group consisting of asthma and rheumatic disease.

142. A method of increasing intestinal absorption of a drug transported by a multi-drug transporter protein comprising administering to a mammal an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

143. A method of improving the penetration of a drug transported by a multi-drug transporter into the central nervous system comprising administering to a mammal an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

144. A method of decreasing renal excretion or renal toxicity of a drug transported by a multi-drug transporter protein comprising administering to a mammal an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

145. A method of treating a malignancy comprising:
(a) administering an effective quantity of an antineoplastic agent transported by a multidrug transporter protein to a mammal with cancer; and
(b) administering an effective amount of a compound having activity against a multi-drug transporter protein, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is —OZ or N(Y₁)-D, where Z is hydrogen, alkyl, aryl, heteroaryl, cycloalkyl, aralkyl, or heteroaralkyl; D is a moiety that promotes absorption of the compound having activity against a multi-drug transporter protein; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

146. The method of claim 145 wherein the antineoplastic agent is selected from the group consisting of adriamycin, etoposide, vinblastine, actinomycin D, and taxol.

147. A method for screening a compound for the existence or nonexistence of multidrug resistance transporter protein inhibitory activity comprising the steps of:
(a) adding the compound to a culture of cancer cells that constitutively express or are induced to express at least one multidrug resistance transporter protein;
(b) adding a cytotoxic agent transported by the multidrug resistance transport protein to the cells;
(c) determining the effect of the compound on the activity of the multidrug resistance transporter protein by performing one or both of a cytotoxicity assay and a drug accumulation assay on the cancer cells to measure either the cytotoxicity of the cytotoxic agent or the accumulation of the cytotoxic agent in the cancer cells; and
(d) comparing the effect of the compound on the activity of the multidrug transporter protein with the effect of a reference compound, N-4-carboxyphenyl-3-(6-oxohydropurin-9-yl)propanamide.
